(19) United States
(12) Patent Application Publication
(10) Pub. No.: US 2017/0209632 A1
(43) Pub. Date: Jul. 27, 2017

(54) APPLICATION-BASED MECHANICAL CIRCULATORY SUPPORT DEVICE ASSESSMENTS

(71) Applicant: Mayo Foundation for Medical Education and Research, Rochester, MN (US)

(72) Inventors: Christopher N. Pierce, Bellingham, WA (US); Lucas A. Schroedl, Phoenix, AZ (US)

(73) Assignee: Mayo Foundation for Medical Education and Research, Rochester, MN (US)

(21) Appl. No.: 15/313,672

(22) PCT Filed: May 27, 2015
(86) PCT No.: PCT/US15/32630

§ 371 Date: Nov. 23, 2016

Related U.S. Application Data
(60) Provisional application No. 62/003,302, filed on May 27, 2014.

(51) Int. Cl. A61M 1/10; A61M 1/12

(52) U.S. Cl. CPC A61M 1/1086; A61M 1/122; A61M 2205/18; A61M 2205/50; A61M 2205/52; A61M 2205/3317; A61M 2205/8206

(57) ABSTRACT

This document describes, among other things, a computer-implemented method that includes receiving, by a computing system, data that represents a plurality of measurements associated with a mechanical circulatory support device connected to a patient over a period of time. The method can include determining, based on the plurality of measurements, respective trends for one or more operating characteristics of the device over the period of time, and generating a respective representation for each of the trends that is formatted for presentation to a user for assessing whether the trends indicate a likelihood of an operational defect associated with the device. The method can include providing the respective representations to the user.

### CROSS REFERENCE TO RELATED APPLICATIONS

This application claims the benefit of U.S. Provisional Ser. No. 62/003,302 filed May 27, 2014. This disclosure of the prior application is considered part of (and is incorporated by reference in) the disclosure of this application.

### TECHNICAL FIELD

This document generally relates to application-based assessments of medical mechanical circulatory support devices such as total artificial hearts and ventricular assist devices.

### BACKGROUND

Treating and managing cardiovascular disease is one of the leading challenges confronting healthcare professionals around the world. Indeed, incidences of heart failure in the United States alone continue to increase each passing year with the number of new cases reported annually recently reaching well over 600,000. For some individuals who are in an advanced stage of heart failure, a total artificial heart (“TAH”) or a ventricular assist device (“VAD”) can be connected to mechanically support blood flow through the individual's circulatory system. TAHs replace an individual's heart organ while VADs complement, rather than replace, an individual's heart organ in instances where the heart is no longer able to adequately or reliably provide circulatory function. In some cases, TAHs and VADs serve as a temporary solution for treating heart failure while patients wait for a heart transplant. In other cases, patients have achieved success with TAHs and VADs for long periods of time lasting many years.

TAHs and VADs are configured to assist or replace the left ventricle, right ventricle, or both ventricles. Native ventricles of a healthy heart accept incoming blood flow through the atrioventricular valves and eject blood to the aorta and pulmonary artery as the ventricles contract during systole. The heart muscle repeatedly expands and contracts causing the ventricles to provide a continuous, pulsating supply of blood. The resulting pressure gradient of this action delivers blood throughout the patient's circulatory system. For patients with various forms of cardiovascular disease, such as congestive heart failure, the heart muscle may be weakened and unable to provide sufficient blood flow to the patient, which can lead to serious risks from inadequate tissue perfusion. In such cases, a TAH or VAD may be used to increase circulation by connecting an intake of the device to the output from the atrioventricular valves, and using a driving unit with an electrical control module to provide either a continuous or pulsatile pressure gradient across the device, thereby overcoming circulatory deficiencies resulting from the weakened ventricles. Several mechanical circulatory support (“MCS”) device types can be used to assist circulation depending on the patient's condition, such as left ventricular assist devices (LVADs), right ventricular assist devices (RVADs), biventricular assist devices (BIVADs), or total artificial hearts (TAHs).

In use, MCS devices may record various information related to the system's operation. For example, the MCS device may include an electric pump that is surgically connected to a patient's circulatory system, but that includes a connection to an externally worn battery pack and computing device for power delivery and system control. Operational information may be collected and electronically stored by the computing device.

### SUMMARY

This document generally describes systems, methods, and other techniques for assessing operational characteristics of mechanical circulatory support devices such as total artificial hearts or ventricular assist devices. The techniques described herein can be used to verify normal system operation, predict device-related clinical pathologies and complications, identify abnormal device behavior, and optimize system performance. These techniques can improve efficacy and overcome limitations of traditional diagnostic modalities such as medical imaging, physical assessment, and blood chemistry analysis. Certain implementations of these techniques can provide one or more advantages. For example, device thrombus can be identified when the patient is asymptomatic or before the patient experiences an adverse event from the thrombus. Moreover, the risk of device-related clinical pathologies and complications can be assessed by using data that is commonly collected by mechanical circulatory support devices such as operating data associated with the system.

In some implementations, a computer-implemented method can include receiving, by a computing system, data that represents a plurality of measurements associated with a mechanical circulatory support device connected to a patient over a period of time. The method can include determining, based on the plurality of measurements, respective trends for one or more operating characteristics of the mechanical circulatory support device over the period of time, and generating a respective representation for each of the trends that is formatted for presentation to a user for assessing whether the trends indicate a likelihood of an operational defect associated with the device or related system. The method can include providing the respective representations to the user.

These and other implementations may include one or more of the following features. The computing system can determine based on the trends, whether there is likely an operational defect associated with the device in the patient.

The computing system can determine that there is likely an operational defect associated with the device, before the patient exhibits physical symptoms from the defect.

Determining whether there is likely an operational defect associated with the device can include comparing a particular one of the determined trends with one or more thresholds that represent acceptable changes in respective operating characteristics of the system over the period of time.

Determining whether there is likely an operational defect associated with the device can include comparing a particular one of the determined trends with a model trend for a corresponding operating characteristic of the device.

The operational defect can include at least one of device-related clinical pathologies and abnormal mechanical circulatory support system behavior.

The mechanical circulatory support device can be a total artificial heart or a ventricular assist device.

The period of time can be at least one second (e.g., 1-5 minutes). The period of time can be at least one hour.

The period of time can be at least one day.

The period of time can be at least one week.

The period of time can be at least one month or at least one year.

Receiving the data that represents the plurality of measurements with the system can include accessing electronic data logs recorded by the device.

The plurality of measurements can include at least one of a current measurement, a power measurement, a voltage measurement, a resistance measurement, an inductance measurement, and a capacitance measurement.

The plurality of measurements can include measurements of electrical characteristics of a driver, such as a driver of a pump motor, in the mechanical circulatory support device. In some implementations, the driver can include a controller that governs operation of the mechanical circulatory support device, and/or can include power-generation circuitry that drives a motor in the mechanical circulatory support device, for example. The measurements can thus include electrical and/or pneumatic characteristics of the a controller, power-generation circuitry, or both.

The plurality of measurements can include measurements of electrical characteristics of a motor in the mechanical circulatory support device.

The plurality of measurements can include measurements of pneumatic characteristics of a driver in the mechanical circulatory support device.

The one or more operating characteristics of the mechanical circulatory support device can represent one or more of power usage by the mechanical circulatory support device or by an electric motor in the mechanical circulatory support device, pulsatility, pulsatility index, ratios of power usage and pulsatility, rate settings, driving pressures, energy utilization, calculation constant settings, and alarms.

Providing the respective representations to the user can include displaying at least a portion of the respective representations on a display of the computing system.

The data that represents the plurality of measurements can be received as having been sent over a network from a computing device remote from the computing system.

In some implementations, one or more computer-readable media can include instructions that, when executed by one or more processors, cause performance of operations. The operations can include receiving, by a computing system, data that represents a plurality of measurements associated with a mechanical circulatory support device connected to a patient over a period of time; determining, based on the plurality of measurements, respective trends for one or more operating characteristics of the mechanical circulatory support device over the period of time; generating a respective representation for each of the trends that is formatted for presentation to a user for assessing whether the trends indicate a likelihood of an operational defect associated with the system; and providing the respective representations to the user.

These and other implementations can optionally include one or more of the following features. The operations can further include determining, by the computing system and based on the trends, whether there is likely an operational defect associated with the device in the patient.

In some implementations, a method for assessing a mechanical circulatory support device can include accessing, from electronic data logs of the mechanical circulatory support device, data that represents a plurality of measurements associated with the mechanical circulatory support device over a period of time, the measurements indicating one or more electrical or pneumatic characteristics of the mechanical circulatory support device during operation of the device in a patient; providing the data that represents the plurality of measurements to a computing system so as to cause generation of respective representations of each of one or more operating characteristics of the mechanical circulatory support device based on the data that represents the plurality of measurements; and determining, based on the respective representations of the one or more operating characteristics, whether trends in particular ones of the operating characteristics over the period of time indicate a likelihood of an operational defect associated with the mechanical circulatory support device.

These and other implementations can optionally include one or more of the following features. The method can further include taking clinical action in response to determining that there is likely an operational defect associated with the mechanical circulatory support device.

The trends can indicate that the mechanical circulatory support device likely has an operational defect, and the particular ones of the operating characteristics can include at least one of power usage by the mechanical circulatory support device, pulsatility, power/pulsatility ratios, rate settings, driving pressures, energy utilization, calculation constant settings, and alarms. In some implementations, the particular ones of the operating characteristics can include at least one of power usage by the mechanical circulatory support device, flow, pulsatility, rate settings, driving pressures, energy utilization, calculation constant settings, alarms, and changes in or ratios of any of these measurements.

### BRIEF DESCRIPTION OF THE DRAWINGS

FIG. 1 is a schematic diagram of an example system for assessing operational characteristics of a medical mechanical circulatory support device.

FIG. 2 is a flowchart of an example process for assessing operational characteristics of a medical mechanical circulatory support device.

FIG. 3A is an example data plot showing trends in mechanical circulatory support device speed as monitored in a particular patient over a period of time.

FIG. 3B is an example data plot showing trends in the mechanical circulatory support device power as monitored in the particular patient over the same period of time shown in FIG. 3A.

FIG. 3C is an example data plot showing trends in the mechanical circulatory support device flow as monitored in the particular patient over the same period of time shown in FIG. 3A.

FIG. 3D is an example data plot showing trends in the mechanical circulatory support device pulsatility index as monitored in the particular patient over the same period of time shown in FIG. 3A.

FIG. 3E is an example data plot showing trends in the mechanical circulatory support device pulsatility/power ratio as monitored in the particular patient over the same period of time shown in FIG. 3A.

FIG. 4 is a schematic diagram of an example computing system that can be used to implement at least some of the techniques described herein.

Like reference numbers represent corresponding parts throughout.

### DETAILED DESCRIPTION

This document generally describes methods, devices, systems, and other techniques for assessing the condition of a mechanical circulatory support (“MCS”) device. Such techniques can be used to determine when there is a likelihood of premature device failure or an operational defect associated with the device, even before problems with the device are manifested clinically. For example, following implantation of a TAH or VAD in a patient, the device may be monitored over a period of time to determine whether trends in one or more operating characteristics of the device indicate early signs of device related clinical pathologies and complications, or abnormal system behavior.

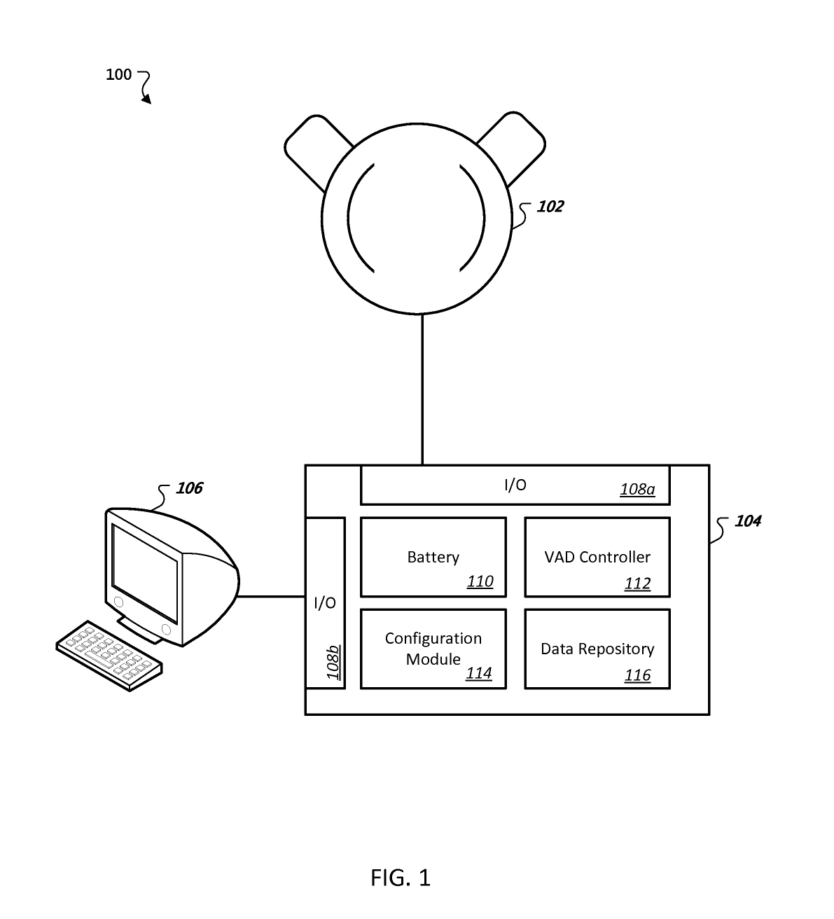

Turning now to FIG. 1, a schematic diagram is shown of an example system 100 for assessing operational characteristics of a medical mechanical circulatory support device. The system 100 includes a VAD 102 (or other MCS device), a control unit 104, and a computing system 106.

The VAD 102 may be surgically connected to a patient in order to provide mechanical circulatory support when the patient's heart is diseased or otherwise weakened and unable to adequately or reliably pump blood through the patient's body. Various types of MCS devices may be employed in the system 100, generally depending on the particular circumstances of the patient's condition. For example, a ventricular assist device can be used to facilitate blood flow from the ventricle. The VAD 102 may, in some examples, be either a left ventricular assist device (“LVAD”), right ventricular assist device (“RVAD”), biventricular assist device (“BIVAD”), or total artificial heart (“TAH”). The VAD 102 may generally provide either pulsatile pumping action or continuous pumping action depending on the particular device.

The VAD 102 communicates with control unit 104, which is adapted to supply the VAD 102 with power and control signals. In some implementations, the control unit 104 can be located outside the patient's body and can communicate with the connected VAD 102 through wired or wireless communication. For example, commercially available MCS devices are often arranged for the patient to wear an electronic unit and/or a battery pack in a harness or other means over the patient's body. In some implementations, one or more of the components of control unit 104 can be provided outside of the patient's body, while one or more other components may be integrated with the VAD 102 or otherwise connected to the patient. Generally, the control unit 104 includes a first I/O interface 108a for bidirectional or unidirectional communication with the connected VAD 102, a second I/O interface 108b for communication with computing system 106, a battery 110, system controller 112, configuration module 114, and data repository 116. The battery 110 may provide primary or backup power to the connected VAD 102.

The system controller 112 can send commands or other signals to the VAD 102 that cause the VAD 102 to operate in a certain manner. For example, in a closed-loop feedback system, the system controller 112 can use information sensed from the VAD 102 or associated sensors to control the speed of the MCS device's motor to circulate blood at a target flow rate. For example, flow sensors or pressure transducers may be connected with the VAD 102, from which information can be sensed for adjusting the speed of the motor in the VAD 102. Other operating parameters can be detected or derived for use by the system controller 112 as well. For instance, control feedback information may be derived from the VAD 102 motor's back electromotive force, from the resistance of the stator or rotor windings, or from electric current levels through the stator or rotor windings. The system controller 112 can work with the configuration module 114 to control the patient's therapy based on a pre-defined therapy profile. For example, the configuration module 114 may include or reference various patient-specific parameters that are stored on non-volatile memory of the control unit 104 and that indicate optimal therapy parameters for the patient. The parameters may be configured by a healthcare professional such as a cardiothoracic surgeon based on an evaluation of the patient's needs. The system controller 112 can then use the parameters to determine a speed of the VAD 102 motor in one example.

The control unit 104 can also include a data repository 116. In some examples, the data repository 116 can be a database or system log file that is stored on nonvolatile memory within the control unit 104. The data repository 116 may include electronic information that is periodically monitored and recorded regarding operation of the VAD 102 within the patient. For example, upon implantation and activation of a VAD 102, software at the VAD 102 or the control unit 104 may be configured to monitor one or more sensors associated with the VAD 102 such as flow sensors or pressure sensors. Electrical characteristics of the VAD 102, such as energy or power usage, resistance, current, back-EMF, and others may also be recorded to track the performance of a device. In some examples, additional information regarding the patient's condition may also be monitored and recorded such as, but not limited to, platelet or fibrinogen levels, LDH levels, bilirubin levels, hemoglobin counts, white blood cell counts, and plasma free hemoglobin counts. Additional information that can also be tracked in conjunction with the aforementioned information includes, but is not limited to, activated partial thromboplastin time, prothrombin time, prothrombin ratio, international normalized ratio, pulsatility, and pulsatility index. Information about medicines administered to the patient in conjunction with the implantation or operation of the VAD 102 may also be recorded within the data repository 116. Some information may be represented directly from recorded sensor data or other measured data, while other information may be derived from measured data through further processing. In some implementations, the information that is tracked and analyzed can include information about any combination of power usage by the mechanical circulatory device, flow, pulsatility, rate settings, driving pressures, energy utilization, calculation constant settings, alarms, and changes in or ratios of any of these measurements. All records stored in the data repository 116 may be associated with respective time stamps that indicate a time when the record was recorded or a time when the underlying measurements for the record were made.

Computing system 106 is adapted to communicate with the control unit 104 to access electronic records from the data repository 116. In some examples, the control unit 104 can transfer all or a portion of the electronic records in data repository 116 to computing system 106. Computing system 106 may be a user device such as a desktop or notebook computer, a tablet computing device, or a smartphone. In some implementations, the computing system 106 can be a server system, a user device connected to a server system, or a networked system of computing devices. The electronic records in the data repository 116 may be device logs that are generally intended to be used and accessed by the manufacturer of the VAD 102 rather than by the patient or his or her health care team.

The computing system 106 can analyze data received from the control unit 104 to assess early indicators of VAD 102 failure. In some implementations, the computing system 106 can analyze the received data and generate representations of the data that indicate trends over a period of time such as a number of days, weeks, or months. Based on the trends, an assessment can be made as to whether the VAD 102 is operating properly or whether there may be problems associated with the internal or external components of device 102. By analyzing such trends, device-related clinical pathologies, complications, or abnormal system behaviors can be identified and resolved, even before the problems have manifested themselves clinically. Indeed, the patient may be completely asymptomatic at the time these problems are identified. For example, trends in operating characteristics of the VAD 102 may indicate early signs of device thrombosis. In the early stages of device thrombosis, the VAD 102 may overcome additional resistance in the circulatory path caused by the thrombosis by drawing more power or requiring more torque to operate at the speed set to achieve a desired blood flow rate. During this period, even though there is a developing problem with the VAD 102, the patient may continue to receive adequate circulation, free of deleterious symptoms. However, the effects of these device-related clinical pathologies, complications, or abnormal device behaviors may become more significant over time and eventually may prevent the VAD 102 from achieving optimal operation. As such, outcomes may be improved by taking remedial action to correct device-related clinical pathologies, complications, or abnormal device behaviors at an early stage as a preventative measure.

In some implementations, trends in power, pulsatility, power/pulsatility ratios, rate settings, driving pressures, energy utilization, calculation constant settings, alarms, and other data or metrics can be used as early indicators of device-related clinical pathologies, complications, or abnormal system behaviors. The computing system 106 can use measured data from the VAD 102 and related components to determine power, pulsatility, power/pulsatility ratios, rate settings, driving pressures, energy utilization, calculation constant settings, alarms and present comparisons of multiple user-defined time intervals over a given time period. Power can reflect a rate of energy consumption by a motor within the VAD 102 at a particular time. Pulsatility is a measure of variation in blood flow through the VAD 102. Rate settings are determined by the end user as the rate at which VAD 102 needs to function to support the patient, and are measured in real-time by the control unit 104. Driving pressures are measurements of the amount of pressure used to drive pulsatile MC S devices. Energy utilization is a product of the external energy used to power the entire MCS system over a given period of time. Calculation constant settings are often used to determine estimated flow and pulsatility parameters through a series of priority calculations created by MCS device manufacturers. Alarms are built in internal algorithms built into the MCS system software to provide the end user with diagnostic information about MCS device parameters or functions that have fallen outside of spec as determined by the manufacturer. As a patient's ventricle repeatedly expands and contracts during the cardiac cycle, blood flow through the VAD 102 fluctuates. Generally, greater blood flow into the ventricle and stronger contractions result in higher pulsatility measurements through the VAD 102. Pulsatility can be represented as a difference between the peak and minimum blood flows through the VAD 102. A closely related measure whose trends may also be analyzed is the pulsatility index, which is a dimensionless measure represented by the formula (Pₘₐₓ−Pₘᵢₙ)/P_{average}, where Pₘₐₓ is the peak (maximum) power consumption through the VAD 102 over of period of time, Pₘᵢₙ is the minimum power consumption through the VAD 102 over the period of time, and P_{average} is the average power consumption over the period of time. Finally, trends in the power/pulsatility ratio can also be used as an early indicator of device-related clinical pathologies, complications, or abnormal system behaviors. For example, an increasing power/pulsatility ratio over time can indicate that an occlusion in the VAD 102, such as an inlet obstruction, is growing more significant. Trends from other ratios may also be used to assess clinical effectiveness and settings optimization of the VAD 102, such as trends in power, pulsatility, power/pulsatility ratios, rate settings, driving pressures, energy utilization, calculation constant settings, and alarms.

In some implementations, data collected or derived from a MCS device can be presented to a qualified health professional in a manner that readily facilitates clinical analysis. For example, a single system may be adapted to pull, analyze, and/or present data from either a particular MCS device or from multiple different types of MCS devices. Thus, a single system may be configured to interact with various models and generations of MCS devices from various manufacturers, and to format or present the data in a similar manner across different types of devices.

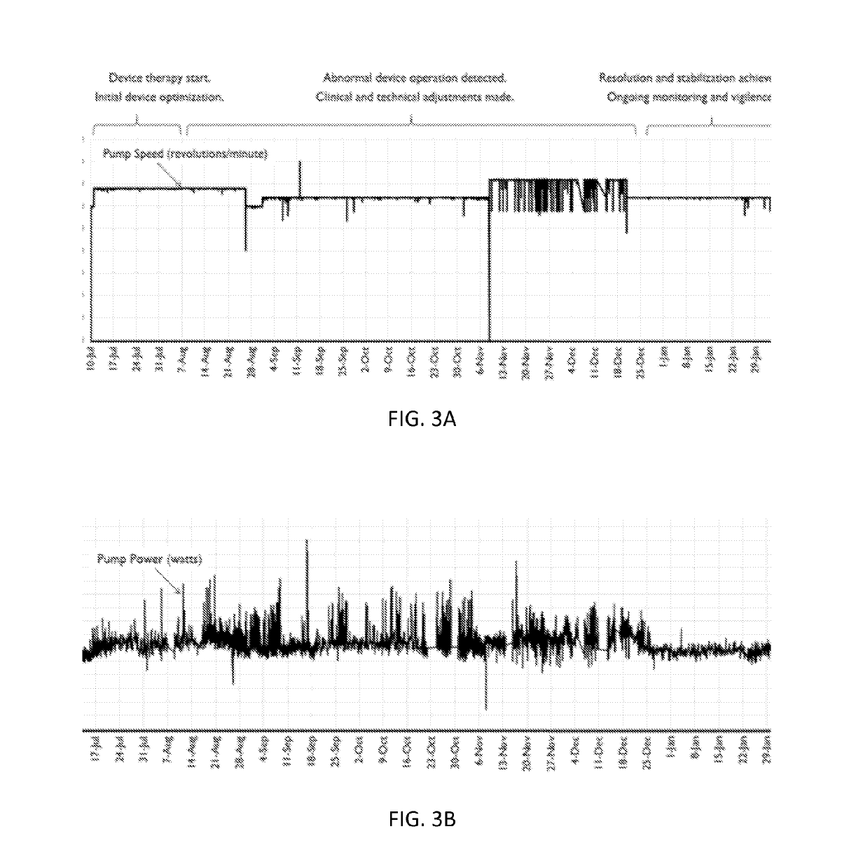

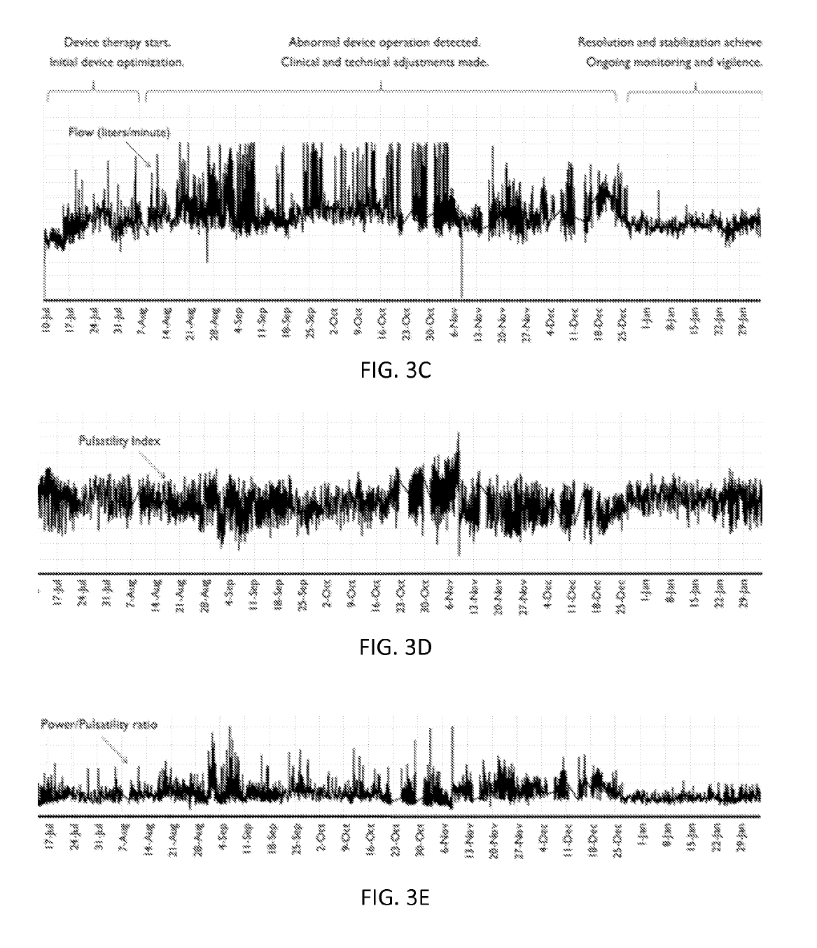

One example of how trends in power, pulsatility, power/pulsatility ratios, rate settings, driving pressures, energy utilization, calculation constant settings, and alarms for a connected VAD 102 can be used to predict device thrombosis is shown in FIG. 3. The top chart in FIG. 3 shows overlapping plots of flow rate and power usage from an actual VAD 102 in an asymptomatic patient over a number of days shortly after implantation and activation of the device. The lower chart is a plot of pulsatility index for the VAD 102 over a corresponding period of time as shown in the top chart. The overall trend shown from the power chart is that the device gradually draws more power over this time, even while the pulsatility index remains in a relatively constant range. This pattern suggests that the VAD 102 may be drawing additional power to overcome resistance from device thrombosis. Removal and inspection of the patient's actual VAD confirmed the thrombosis.

The computing system 106 can facilitate identification and analysis of trends in the VAD 102 operating characteristics. In some implementations, the computing system 106 can compute one or more values that quantify the trends. For example, the computing system 106 may compute one or more statistical values such as a mean or standard deviation for the data. Statistical values can also be computed for multiple groups of data that correspond to data within different portions of time of the overall time period for which trends are being analyzed. Thus, for example, respective average power values can be calculated for initial, middle, and end portions of time, and trends may be quantified based on the changes between the values for each portion of time. Other more sophisticated statistical analyses can also be performed, such as regression techniques that identify a “best-fit” line to model the data so that the trends may be more clearly represented. In some implementations, the computing system 106 can generate plots for VAD 102 operating data and display the plots, along with relevant statistical information, to a user. The user, such as a qualified healthcare professional, may use the computer-generated representation of data to analyze the trends and make an informed assessment as to whether there are any problems associated with the VAD 102, such as indications of clinical pathology or device malfunction. In some implementations, the computing system 106 may perform statistical analyses to identify trends in operating data and determine a likelihood of such problems without user interaction. The computing system 106 may also indicate a confidence level for the prediction that quantifies the likelihood or risk of a problem with the VAD 102. The computing system 106 may perform statistical analysis on the data and determine a likelihood or risk of a problem with the VAD 102 based on preset or user-defined configurations. For example, a user may select the window of time over which the analysis is to be performed.

In certain implementations, the computing system 106 may access applications or services that perform all or a portion of the operations described herein such as data processing, statistical analysis, data representation, user visualization, thrombosis prediction, and other operations for assessing a MCS device. The applications or services may be installed on the computing system 106, or may be web applications or other applications accessed from a central server system. In some examples, the applications or services may be cloud-based applications that are accessible by various computing devices over a network connection. The applications or services may be programmed to allow for drag-and-drop operations. For example, upon establishing a connection between control unit 104 and computing system 106, a user can select the data logs that he or she wishes to analyze, and the computing system 106 can automatically analyze the data. The applications or services may be distributed or otherwise made accessible to a plurality of health care providers in one or more locations so that the techniques described herein for early assessment of MCS device operations can be implemented as a standard of care for the health care providers.

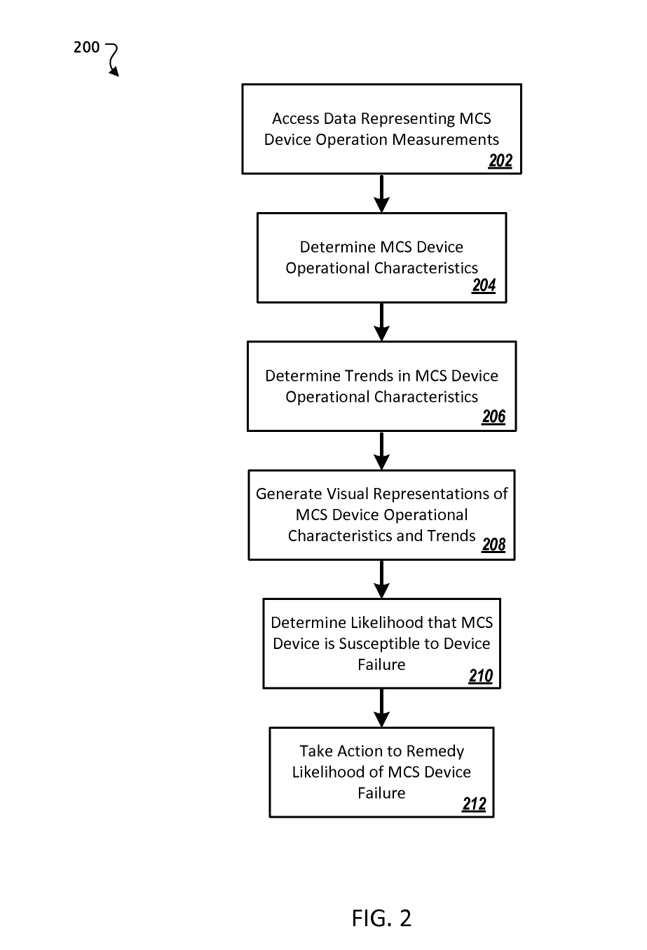

FIG. 2 is a flowchart of an example process 200 for assessing operational characteristics of a MCS device, such as a VAD. In some implementations, the process 200 may be performed by one or more components of the system 100 depicted in FIG. 1.

At operation 202, the process 200 includes accessing data that represents operational measurements associated with a MCS device. The data may be recorded and stored by an internal component or an associated external component such as an external control unit. In some examples, the accessed data may include multiple entries that are each associated with a particular time and that indicate a current operating condition of the MCS device or a condition of the patient. The data may reflect either raw or processed measurements from one or more sensors associated with the MCS device. For example, sampled data from flow sensors or pressure transducers within the device or the patient's circulatory path may be recorded. Electrical characteristics associated with the system such as voltages, current, resistance, and power usage by the system at particular times may be recorded. The patient's MCS device may be configured to monitor and record such data periodically.

At operation 204, the process 200 includes determining operational characteristics associated with the MCS device. Information indicating operational characteristics can be derived from the data accessed from device logs in operation 202. For example, the device logs may include entries corresponding to raw flow sensor data, motor electrical current and back-emf voltages, among other data. At operation 204, the raw data can be processed to determine one or more operational characteristics of the MCS device that can be used, for example, in assessing a likelihood of component malfunction. In some implementations, the data can be processed to determine trends in power, pulsatility, power/pulsatility ratios, rate settings, driving pressures, energy utilization, calculation constant settings, and alarms. Trends in these operational characteristics can then be analyzed to determine a likelihood of system optimization. The process 200 may calculate intermediate values in arriving at one or more of the operational characteristics. For instance, maximum, minimum, and average flow rates may be determined for use in calculating pulsatility index values.

At operation 206, trends are determined from the operating characteristics. Trends may be determined over any period of time for which data is available. The period of time may be automatically determined by a computing system, or may be manually selected. In some instances, trends may be determined over multiple different time periods with or without overlap to determine how the trends may vary among the different time periods. Generally, the period of time may be over multiple days or weeks and sufficiently long to confidently determine a trend, and may be based on expected development rates associated with normal system operation, various clinical pathologies or device abnormalities. Trends may be quantified by a computing system using statistical analysis techniques, or may be displayed, for example by plotting the operating characteristics over the relevant period of time, to enable a qualified healthcare professional to visualize the trends. Thus, the process 200 can include, at operation 208, generating visual representations of the operating characteristics for trend analysis.

In some implementations, data that has been collected or processed relating to a MCS device or patient with a MCS device may be personalized to particular patients. For example, baseline measurements may be determined for a particular patient, and deviations away from that patient's baseline, such as over a time period sufficiently long to indicate a trend, may be used to inform a qualified healthcare professional of a likelihood of clinical pathology or abnormal system operation.

At operation 210, a determination is made about potential defects or other problems associated with the MCS device. Based on the trends in operating characteristics, a likelihood of clinical pathology or abnormal system operation in a MCS device may be determined. For instance, a developing motor abnormality can reduce operating efficiency and present erratic power and speed signatures. The MCS device may draw increasingly more power and demonstrate inability to maintain set rate, which may be reflected in the trends in the operational characteristics. For example, gradually increasing power requirements to maintain relatively constant set rate or unexpected supranormal pulsatility likely indicates deterioration of the MCS system driveline component.

At operation 212, appropriate clinical action can be taken based on the determinations about potential defects or other problems associated with the connected MCS device. For example, if it is determined that there is a significant risk from abnormal MCS system operation, then qualified healthcare professionals may act on this information before the condition manifests itself clinically in the patient. The techniques described in this document can enable early identification of device-related clinical pathologies and abnormal MCS system behavior even when the patient is asymptomatic and before the occurrence of adverse clinical events. Acting upon such information, the patient may have MCS system components adjusted, repaired or replaced, be administered appropriate medications, or be referred for surgery to replace or remove the device.

FIGS. 3A-3E depict example data plots of trends in MCS device-related parameters that were monitored in a particular patient over a period of 7 months. The plots in FIGS. 3A-3E show, respectively, device speed (revolutions/minute), power (watts), flow (liters/minute), pulsatility index (dimensionless), and power to pulsatility ratio (a dimensionless calculation based on power and pulsatility index) over time. The time axis is scaled similarly among all the plots, while each of the plots has its own y-axis according to the above-mentioned units in a respective range.

The plots in FIGS. 3A-3E show a narrow range of power and flow in the early stages of device initiation and optimization, with an early abrupt increase in quantity and variability of both power and flow. Concomitantly, the pulsatility index is noted to decrease, causing an increase in calculated power/pulsatility ratio. Changes of this nature and magnitude in the early device therapy time frame are unusual and should raise clinical suspicion of building subclinical pathology, even in absence of physical signs and symptoms, normal blood chemistry values, or device operation within manufacturer's normal limits. In some implementations, the determined trends can be highly sensitive, uniquely descriptive of device performance, and can predict device prognosis. For example, for the particular patient whose device-related parameters are depicted in FIGS. 3A-3E, the system accurately predicted the trajectory of device disposition of this case as power, flow, and pulsatility index plateaued for the next several weeks, but with high variability. The patient presented with clinical stability and diminishing heart failure symptoms, but mildly abnormal blood chemistry values. The high variability of monitored device parameters can be indicative of abnormal device operation but was imperceptible during conventional device monitoring. Other techniques were unsuccessful in detecting the abnormal device operation. The patient's mildly abnormal blood chemistry values corroborated the technical assessment.

The abnormal device operation was addressed with clinical and technical adjustments. Monitored device parameters demonstrated persistent high variability with brief improvement periods during the clinical and technical adjustments. This analysis method confirmed that clinical and technical adjustments were unsuccessful. There was no resolution of the patient's abnormal blood chemistry values, also. The device was surgically exchanged and a significant thrombus was found on the distal rotor bearing and outlet stator. Post-exchange analysis demonstrated resolution of abnormal device operation with normalization of device variability and stabilization of power, flow, pulsatility index, and the power/pulsatility ratio.

The use of this analysis system led the medical team to follow an aggressive therapy plan. Because of the acceptable blood chemistry analysis and clinical presentation of the patient, this issue likely would have otherwise proceeded unnoticed with potential for progression into a significantly morbid or possible mortal event. In some implementations, the methods, systems, and other techniques described in this paper offer advantages in that MCS device-related parameters and trends determined therefrom can be used as a reliable and reproducible diagnostic and management tool for guiding medical professionals in their clinical practices by improving patient survival, demonstrating adequacy of clinical methods, and predicting therapy outcome.

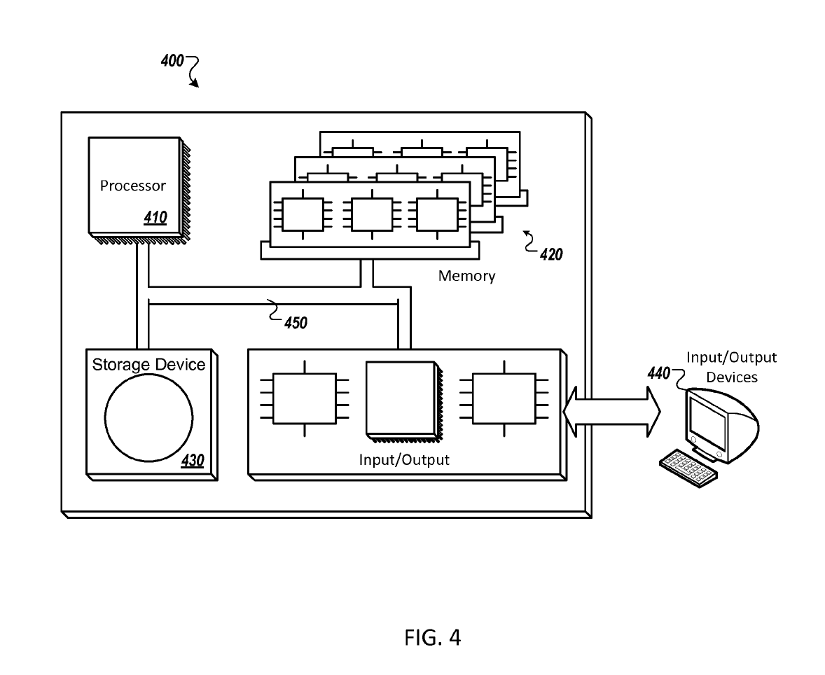

FIG. 4 is a schematic diagram of a computer system 400. The system 400 can be used for the operations described in association with any of the computer-implemented methods described previously, according to one implementation. The system 400 is intended to include various forms of digital computers, such as laptops, desktops, workstations, personal digital assistants, servers, blade servers, mainframes, and other appropriate computers. The system 400 can also include mobile devices, such as personal digital assistants, cellular telephones, smartphones, and other similar computing devices. Additionally the system can include portable storage media, such as, Universal Serial Bus (“USB”) flash drives. For example, the USB flash drives may store operating systems and other applications. The USB flash drives can include input/output components, such as a wireless transmitter or USB connector that may be inserted into a USB port of another computing device.

The system 400 includes a processor 410, a memory 420, a storage device 430, and an input/output device 440. Each of the components 410, 420, 430, and 440 are interconnected using a system bus 450. The processor 410 is capable of processing instructions for execution within the system 400. The processor may be designed using any of a number of architectures. For example, the processor 410 may be a CISC (Complex Instruction Set Computers) processor, a RISC (Reduced Instruction Set Computer) processor, or a MISC (Minimal Instruction Set Computer) processor.

In one implementation, the processor 410 is a single-threaded processor. In another implementation, the processor 410 is a multi-threaded processor. The processor 410 is capable of processing instructions stored in the memory 420 or on the storage device 430 to display graphical information for a user interface on the input/output device 440.

The memory 420 stores information within the system 400. In one implementation, the memory 420 is a computer-readable medium. In one implementation, the memory 420 is a volatile memory unit. In another implementation, the memory 420 is a non-volatile memory unit.

The storage device 430 is capable of providing mass storage for the system 400. In one implementation, the storage device 430 is a computer-readable medium. In various different implementations, the storage device 430 may be a floppy disk device, a hard disk device, an optical disk device, or a tape device.

The input/output device 440 provides input/output operations for the system 400. In one implementation, the input/output device 440 includes a keyboard and/or pointing device. In another implementation, the input/output device 440 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer. Additionally, such activities can be implemented via touchscreen flat-panel displays and other appropriate mechanisms.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

While this specification contains many specific implementation details, these should not be construed as limitations on the scope of any inventions or of what may be claimed, but rather as descriptions of features specific to particular implementations of particular inventions. Certain features that are described in this specification in the context of separate implementations can also be implemented in combination in a single implementation. Conversely, various features that are described in the context of a single implementation can also be implemented in multiple implementations separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

Thus, particular implementations of the subject matter have been described. Other implementations are within the scope of the following claims. In some cases, the actions recited in the claims can be performed in a different order and still achieve desirable results. In addition, the processes depicted in the accompanying figures do not necessarily require the particular order shown, or sequential order, to achieve desirable results. In certain implementations, multitasking and parallel processing may be advantageous.

What is claimed is:

1. A computer-implemented method comprising:
receiving, by a computing system, data that represents a plurality of measurements associated with a mechanical circulatory support device connected to a patient over a period of time;
determining, based on the plurality of measurements, respective trends for one or more operating characteristics of the mechanical circulatory support device over the period of time;
generating a respective representation for each of the trends that is formatted for presentation to a user for assessing whether the trends indicate a likelihood of an operational defect associated with the mechanical circulatory support device; and
providing the respective representations to the user.

2. The computer-implemented method of claim 1, further comprising determining, by the computing system and based on the trends, whether there is likely an operational defect associated with the device in the patient.

3. The computer-implemented method of claim 2, wherein the computing system determines that there is likely an operational defect associated with the device, and wherein the determination as to whether there is likely an operational defect is made before the patient exhibits symptoms from the defect.

4. The computer-implemented method of claim 2, wherein determining whether there is likely an operational defect associated with the device includes comparing a particular one of the determined trends with one or more thresholds that represent acceptable changes in respective operating characteristics of the device over the period of time.

5. The computer-implemented method of claim 2, wherein determining whether there is likely an operational defect associated with the device includes comparing a particular one of the determined trends with a model trend for a corresponding operating characteristic of the device.

6. The computer-implemented method of claim 2, wherein the operational defect comprises at least one of device-related clinical pathologies and abnormal mechanical circulatory support device system behavior.

7. The computer-implemented method of claim 1, wherein the mechanical circulatory support device comprises a total artificial heart or a ventricular assist device.

8. The computer-implemented method of claim 1, wherein the period of time is at least one hour.

9. The computer-implemented method of claim 1, wherein the period of time is at least one day.

10. The computer-implemented method of claim 1, wherein the period of time is at least one week.

11. The computer-implemented method of claim 1, wherein the period of time is at least one month.

12. The computer-implemented method of claim 1, wherein receiving the data that represents the plurality of measurements with the device comprises accessing electronic data logs recorded by the device.

13. The computer-implemented method of claim 1, wherein the plurality of measurements include at least one of a current measurement, a power measurement, a voltage measurement, a resistance measurement, an inductance measurement, and a capacitance measurement.

14. The computer-implemented method of claim 1, wherein the plurality of measurements include measurements of electrical and pneumatic characteristics of a driver in the device.

15. The computer-implemented method of claim 1, wherein the plurality of measurements include measurements of electrical characteristics of a motor in the device.

16. The computer-implemented method of claim 1, wherein the plurality of measurements include measurements of pneumatic characteristics of a driver in the device.

17. The computer-implemented method of claim 1, wherein the one or more operating characteristics of the device represent one or more of power usage by the device or by an electric motor in the device, pulsatility, pulsatility index, ratios of power usage and pulsatility, rate settings, driving pressures, energy utilization, calculation constant settings, and alarms.

18. The computer-implemented method of claim 1, wherein providing the respective representations to the user comprises displaying at least a portion of the respective representations on a display of the computing system.

19. The computer-implemented method of claim 1, wherein the data that represents the plurality of measurements is received as having been sent over a network from a computing device remote from the computing system.

20. One or more computer-readable media including instructions that, when executed by one or more processors, cause performance of operations comprising:
receiving, by a computing system, data that represents a plurality of measurements associated with a mechanical circulatory support device connected to a patient over a period of time;
determining, based on the plurality of measurements, respective trends for one or more operating characteristics of the device over the period of time;
generating a respective representation for each of the trends that is formatted for presentation to a user for assessing whether the trends indicate a likelihood of an operational defect associated with the device; and
providing the respective representations to the user.

21. The computer-readable media of claim 20, wherein the operations further comprise determining, by the computing system and based on the trends, whether there is likely an operational defect associated with the device in the patient.

22. A method for assessing a mechanical circulatory support device comprising:
accessing, from electronic data logs of the device, data that represents a plurality of measurements associated with the device over a period of time, the measurements indicating one or more electrical or pneumatic characteristics of the device during operation of the device in a patient;
providing the data that represents the plurality of measurements to a computing system so as to cause generation of respective representations of each of one or more operating characteristics of the device based on the data that represents the plurality of measurements; and
determining, based on the respective representations of the one or more operating characteristics, whether trends in particular ones of the operating characteristics over the period of time indicate a likelihood of an operational defect associated with the device.

23. The method of claim 22, further comprising taking clinical action in response to determining that there is likely an operational defect associated with the mechanical circulatory support device.

24. The method of claim 22, wherein the trends indicate that the mechanical circulatory support device likely has an operational defect, and wherein the particular ones of the operating characteristics include at least one of power usage by the device, pulsatility, power/pulsatility ratios, rate settings, driving pressures, energy utilization, calculation constant settings, and alarms.
